(19) United States
(12) Patent Application Publication
(10) Pub. No.: US 2006/0246131 A1
(43) Pub. Date: Nov. 2, 2006

(54) Use of metformin to counteract weight gain associated with psychotropic medications

(76) Inventor: Elizabeth M. Cottlingham, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 11/407,231

(22) Filed: Apr. 19, 2006

Related U.S. Application Data
(60) Provisional application No. 60/675,534, filed on Apr. 28, 2005.

(51) Int. Cl. A61K 31/519; A61K 31/496; A61K 31/325; A61K 9/20

(52) U.S. Cl. 424/464; 514/259.41; 514/253.07; 514/477

(57) ABSTRACT

A method for minimizing the weight gain side effect associated with ABILIFY® (aripiprazole) or GEODON® (ziprasidone) treatment is disclosed. In this method, metformin, a biguanide compound, is concurrently administered to a patient taking the ABILIFY® (aripiprazole) or GEODON® (ziprasidone) therapy. A pharmaceutical composition containing the combination of ABILIFY® (aripiprazole) or GEODON® (ziprasidone), together with metformin is also disclosed.

### CROSS-REFERENCE TO RELATED APPLICATION

The present application is related to and claims priority from U.S. Provisional Patent Application No. 60/675,534, Cottingham, filed Apr. 28, 2005, incorporated herein by reference.

### TECHNICAL FIELD

The present invention relates to improvements in the treatment of patients for schizophrenia, bipolar disorder, psychosis and other psychiatric illnesses.

### BACKGROUND OF THE INVENTION

ABILIFY® (aripiprazole) is a psychotropic drug that is available in tablet form for oral administration. It functions as a dopamine partial agonist and, as a result of this unique mechanism, is thought to be different from the other atypical antipsychotic drugs.

GEODON® (ziprasidone) is also a psychotropic drug available as capsules for oral administration. It is chemically unrelated to phenothiazine or butyrophenone antipsychotic agents.

In general, the known antipsychotic agents cause weight gain in the patients taking them. This can be a difficult side effect to deal with, since it can easily result in non-compliance by the patient (i.e., the patient stops taking the drug or takes it at reduced frequency) leading to major problems. Although ABILIFY® and GEODON® were thought to result in reduced weight gain seen, there is still significant weight gain, at least in some patients. For example, Jaworowski, S. et al., Ziprasidone and Weight Gain, Clin. Neuropharmacol. 2004 Mar.-Apr. 27(2):99-100, reported significant weight gain in a 12-year-old male treated with GEODON®.

Clearly, from both a compliance and a patient self-esteem point-of-view, it would be very helpful to eliminate or minimize the weight gain caused by ABILIFY® and GEODON®.

Metformin is a biguanide drug which is known to improve insulin action at the cellular level, but not affect insulin secretion. Metformin is used to treat patients with non-insulin dependent diabetes and has recently been used to treat women with polycystic ovary syndrome, a syndrome characterized by hirsutism, hyperandrogenism, and polycystic ovaries. It has not, however, been suggested for use in controlling the weight gain caused by ABILIFY® or GEODON®. See, for example, Valazquez, et al, Metformin Therapy Is Associated With A Decrease In Plasma Plasminogen Activator Inhibitor-1, Lipoprotein (a) and Immunoreactive Insulin Levels In-Patients With Polycystic Ovary Syndrome. Metabolism, 46: 454-457 (1997); Valazquez, et al, Metformin Therapy In Polycystic Ovary Syndrome Reduces Hyperinsulinemia, Insulin Resistance, Hyperandrogenism, And Systolic Blood Pressure, While Facilitating Normal Menses And Pregnancy. Metabolism, 43: 647-654 (1994); Jackson, et al., Mechanism of Metformin Action In Non-Insulin Dependent Diabetes. Diabetes; 36: 632-640 (1987); Landin, et al., Treating Insulin Resistance in Hypertension With Metformin Reduces Both Blood Pressure And Metabolic Risk Factors. J. Intern. Med.; 229: 181-187 (1991); and Nestler, et al., Effects of Metformin on Spontaneous and Clomiphene-Induced Ovulation in the Polycystic Ovary Syndrome. N. Engl. J. Med. 338: 1876-1880 (1998).

The use of metformin to counteract weight gain in patients caused by the psychotropic actives DEPAKOTE® (valproate), RESPERDAL®, LITHOBID®, ZYPREXA® and SEROQUEL® is taught in U.S. Pat. No. 6,194,466, Cottingham and Morrison, issued Feb. 27, 2001.

### SUMMARY OF THE INVENTION

The present invention relates to a method for minimizing weight gain in a patient taking a psychotropic active selected from the group consisting of ABILIFY® (aripiprazole) and GEODON® (ziprasidone), comprising the administration to said patient of a safe and effective amount of metformin or a similar compound.

The present invention also encompasses a combination drug composition which comprises a safe and effective amount of a psychotropic active select from the group consisting of ABILIFY® (aripiprazole) and GEODON® (ziprasidone), together with a safe and effective amount of metformin or a similar compound

### DETAILED DESCRIPTION OF THE INVENTION

The present invention relates to a method for minimizing weight gain in a patient taking the antipsychotic medications ABILIFY® and GEODON®.

ABILIFY® (aripiprazole) is an antipsychotic drug that is available as tablets for oral administration. Aripiprazole is 7-[4-[4-(2,3-dichlorophenyl)-1-piperazinyl]butoxy]-3,4-dihydrocarbostyril. Its empirical formula is C₂₃H₂₇Cl₂N₃O₂ and its molecular weight is 448.38. The structural formula of the compound is

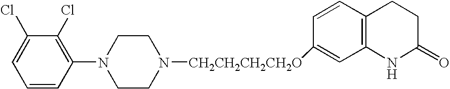

Aripiprazole is chemically different from other atypical antipsychotic agents and is also believed to have unique pharmacological actions that are different from other atypical antipsychotic drugs, including ZYPREXA®, SEROQUEL® and RISPERDAL®. Aripiprazole acts as a weak stimulator (so-called “partial” agonist) at dopamine D₂ receptors, with the potential for exerting either antagonistic (inhibitory) or agonistic (stimulating) effects, depending on the sensitivity of the receptors and the availability of dopamine, its natural agonist in the brain. Aripiprazole also has similar activity at serotonin-5-HT_{1A} receptors, as well as acting as an antagonist at serotonin-5-HT_{2A} receptors, and having a number of other lesser actions.

GEODON® is available as GEODON® capsules (ziprasidone hydrochloride) for oral administration. Ziprasidone is an antipsychotic agent that is chemically unrelated to phenothiazine or butyrophenone antipsychotic agents. It has an empirical formula of C₂₁H₂₁ClN₄OS (free base) and a molecular weight of 412. Its chemical name is 5-[2-[4-(1,2-benzisothiazol-3-yl)-1-piperazinyl]-6-chloro-1,3-dihydro-2H-indol-2-one; and the following structural formula

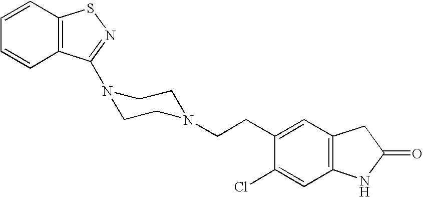

Other pharmaceutically acceptable salts of both of these actives may also be used herein.

Although they are both effective antipsychotic agents, both ABILIFY® and GEODON® cause some degree of weight gain in many patients. It is that effect which the present invention addresses.

Metformin hydrochloride is a biguanide compound that is generally prepared as an oral anti-hyperglycemic drug used in the management of non-insulin-dependent diabetes mellitus. It is typically prepared in the form of tablets and is commercially available as GLUCOPHAGE® from the Bristol-Myers Squibb Company. It is also available as a liquid for oral administration. Metformin hydrochloride (N,N-dimethylimidocarbonimidic diamide hydrochloride) has the structural formula shown below:

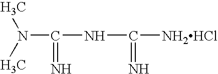

In addition to metformin hydrochloride, other pharmaceutically acceptable salts of metformin may be used. Metformin hydrochloride is a white to off-white crystalline compound with a molecular formula of C₄H₁₁N₅.HCl, and a molecular weight of 165.63. Metformin hydrochloride is freely soluble in water and is practically insoluble in acetone, ether or chloroform. The pKa of metformin is 12.4. The pH of a 1% aqueous solution of metformin hydrochloride is 6.68. GLUCOPHAGE® tablets contain 500 mg or 850 mg of metformin hydrochloride. In addition, each tablet contains the following inactive ingredients: povidone, magnesium stearate and hydroxypropyl methylcellulose coating.

The metformin dosage forms used in the present invention optionally may be formulated for controlled release, sustained release, delayed release, or response release (i.e., the tablet is ingested and the active is released in response to the occurrence of a precondition in the patient, such as the intake of food by the patient, or changes in pH, sugar levels or osmolarity in the patient).

In practicing the method of treatment of the present invention, metformin (or another pharmaceutically acceptable salt of N,N-dimethylimidocarbonimidic diamide) is administered to a patient on ABILIFY® or GEODON® therapy. The psychotropic actives will be administered using their conventional routes of administration and their conventional dosage levels. Metformin may be administered to the patient in any way known in the art, although oral administration will generally be most convenient. Metformin is administered in an amount that is safe and effective for minimizing the weight gain associated with ABILIFY®/GEODON® therapy, preferably at a level of from about 1500 to about 2500 mg per day. It is typically administered with meals at a dosage of 500 mg tid.

The present invention also encompasses a combination drug that includes the active found in ABILIFY® or GEODON® (or other pharmaceutically acceptable salts), together with metformin or other biguanide compounds (including other pharmaceutically acceptable salts of N,N-dimethylimidocarbonimidic diamide). This combination of drugs is typically formulated as a tablet or capsule for oral administration, although other routes of administration, such as intravenous injection can also be used. A tablet or capsule for oral administration of the present invention would typically include from about 2.5 mg to about 30 mg of ABILIFY® or from about 20 mg to about 80 mg GEODON®, and from about 250 mg to about 850 mg of metformin. Conventional formulational aides, such as fillers, coatings, preservatives, disintegration aides, colorings and flavorings, can also be included at their conventional art-established levels.

By “pharmaceutically acceptable,” as used herein, is meant that the drug-active compounds and other ingredients used in the present methods and compositions, are suitable for use in contact with the tissues of humans without undue toxicity, irritation, allergic response, and the like, commensurate with a reasonable benefit/risk ratio.

### CASE STUDY

Presentation & Diagnosis

A 10-year-old female presented with behavioral problems. The patient had an extensive history of behavioral problems, including issues with authority figures, problems with anger modulation and management, and instances of explicit sexual behavior.

The patient was adopted, and she lived with her adoptive parents and her non-biological sister. Academically, she was reported to be doing well, she had positive peer relations, and was involved in cheerleading and sporting activities such as basketball and softball.

On presentation, the patient's parents were primarily concerned with her mood labiality. They observed that she could go from “sweet” to “angry” on a daily basis. She had episodes of nocturnal restlessness; she exhibited difficulty concentrating, and was both disorganized and inattentive. Her energy level was described as appropriate, however she had a history of aggressive and destructive behavior. For example, she peeled wallpaper off the walls, broke blinds, and damaged spindles on the stairs. She had physically attacked her mother, leaving scratches and bruises. She was reported to be oppositional and defiant, calling her mother names and refusing to abide by the rules and regulations of the household. She had also been caught stealing money within the home.

During the psychiatrist's interview, the patient reported feeling angry with her parents about scheduling an appointment, and was unhappy in general. While initially uncooperative, she participated more as the evaluation progressed. She demonstrated a lack of insight regarding her behaviors and overall poor judgment. However, she denied suicidal or homicidal ideation, or any symptoms of psychosis.

Diagnosis

The patient's presentation was strongly suggestive of Bipolar Affective Disorder, Not Otherwise Specified (BPAD NOS). The time of onset for childhood BPAD is frequently during the later years of the latency period, and typically includes rapid cycling, with disturbances in sleep, behavioral issues including aggression, grandiosity, and hyper-sexuality. At the time of evaluation, it was difficult to know if the patient also had co-occurring ADHD, combined subtype and Oppositional Defiant Disorder. Bipolar Affective Disorder, NOS appeared to be the more parsimonious diagnosis.

Treatment and Outcome

Given the complexity of making a diagnosis of BPAD in children and adolescents, the parents were encouraged to read about BPAD and treatment with medication. Additionally they were asked to keep a mood chart. On their return visit, the parents agreed that the patient's symptoms were most consistent with BPAD, and believed that she primarily had manic symptoms. Various medications were discussed to achieve mood stabilization in the patient, including lithium, divalproex, various anticonvulsants, and the atypical antipsychotics. After full discussion of the risks, side effects and advantages of each medication, including concerns about frequent laboratory monitoring, weight gain and end organ effects, she was started on aripiprazole (5 mg, once daily). Parents were also directed to seek psychotherapy for the patient and the family, to assist with the management of her BPAD.

On a return visit 2 weeks later, the patient was reportedly much more settled, with fewer outbursts, and no behavioral concerns. The patient continued to do well on arpiprazole, but she gained weight: having started the treatment at 78 lbs, increasing to 100 lbs in approximately six months.

The patient was started on metformin (titrated to 500 mg, twice daily) together with the aripiprazole, to remediate the weight gain. She tolerated the metformin without side effects, and she exhibited a subsequent decrease in her Body Mass Index (BMI), from 24.9 to 22.6, over a 3-month period, a decrease of seven pounds. The patient continued on this regimen and had no additional concerns about her weight.

### REFERENCES

Management options for bipolar disorder in children and adolescents.
Journal: Paediatr Drugs (Author: Danielyan, A; Kowatch, R A; Year: 2005; Vol: 7(5); Pages: 277-297)

Prevalence of atypical antipsychotic drug use among commercially insured youths in the United States.
Journal: Arch Pediatr Adolesc Med (Author: Curtis, L H; Masselink, L E; Ostbye, T; Year: 2005; Vol: 159(4); Pages: 362-366)

Weight change with atypical antipsychotics in the treatment of schizophrenia.
Journal: J Psychopharmacol (Author: Haddad, P; Year: 2005; Vol: 19(6); Pages: 16-27)

Metformin for weight loss in pediatric patients taking psychotropic drugs.
Journal: Am J Psychiatry (Author: Morrison, J A; Cottingham, E M; Barton, B A; Year: 2002; Vol: 159(4); Pages: 655-657)

Options for pharmacological management of obesity in patients treated with atypical antipsychotics.
Journal: Int Clin Psychopharmacol (Author: Werneke, U; Taylor, D; Sanders, T A; Year: 2002; Vol: 17(4); Pages: 145-160)

Clinical experience with Topiramate to counteract neuroleptic induced weight gain in 10 individuals with autistic spectrum disorders.
Journal: Brain Dev (Author: Canitano, R; Year: 2005; Vol: 27(3); Pages: 228-232)

A Double-Blind, Placebo-Controlled Trial of Sibutramine for Olanzapine-Associated Weight Gain.
Journal: Am J Psychiatry (Author: Henderson, D C; Copeland, P M; Daley, T B; Year: 2005; Vol: 162(5); Pages: 954-962)

Double blind, placebo-controlled investigation of amantadine for weight loss in subjects who gained weight with olanzapine.
Journal: Amer J of Psychiatry (Author: Graham, K A; Gu, H; Lieberman, J A; Year: 2005; Vol: 162; Pages: 1744-1746)

What is claimed is:

1. A method for minimizing weight gain in a patient taking a psychotropic active selected from the group consisting of aripiprazole and ziprasidone, comprising the administration to said patient of a safe and effective amount of a weight control active compound comprising a pharmaceutically-acceptable salt of N,N-dimethylimidocarbonimidic diamide

2. The method according to claim 1 wherein the weight control active is the hydrochloride salt.

3. The method according to claim 2 wherein the psychotropic active is aripiprazole.

4. The method according to claim 3 wherein the weight control active is administered orally.

5. The method according to claim 4 wherein the weight control active is administered in an amount of from about 1500 mg to about 2500 mg per day.

6. A pharmaceutical composition comprising a safe and effective amount of a psychotropic active selected from the group consisting of aripiprazole and ziprasidone, together with a weight control active comprising a pharmaceutically-acceptable salt of N,N-dimethylimidocarbonimidic diamide in an amount safe and effective for minimizing weight gain caused by said psychotropic active in the patient taking said psychotropic active.

7. The composition according to claim 6 wherein the weight control active is the hydrochloride salt.

8. The composition according to claim 7 wherein the psychotropic active is aripiprazole.

9. The composition according to claim 8 which is formulated for oral administration.

10. The composition according to claim 9 which contains from about 2.5 mg to about 30 mg aripiprazole, and from about 250 mg to about 850 mg of the weight control active.

11. The composition according to claim 6 wherein the psychotropic active is ziprasidone present at from about 20 mg to about 80 mg, and from about 250 mg to about 850 mg of the weight control active.

12. Use of a pharmaceutically-acceptable salt of N,N-dimethylimidocarbonimidic diamide for manufacture of a composition for use in minimizing weight gain in a patient taking a psychotropic active selected from the group consisting of aripiprazole and ziprasidone.

13. The use according to claim 12 wherein the pharmaceutically acceptable salt is the hydrochloride salt.
